SEQUENCE LISTING (20 sequences)

SEQ ID NO: 1 - protein, 20 aa, synthetic construct
PVSQPDAPGHQRKVVSWIDV

SEQ ID NO: 2 - protein, 20 aa, synthetic construct
SWIDVYTRATCQPREVVVPL

SEQ ID NO: 3 - protein, 20 aa, synthetic construct
VVVPLTVELMGTVAKQLVPS

SEQ ID NO: 4 - protein, 20 aa, synthetic construct
QLVPSCVTVQRCGGCCPDDG

SEQ ID NO: 5 - protein, 20 aa, synthetic construct
CPDDGLECVPTGQHQVRMQI

SEQ ID NO: 6 - DNA, 18 bp, synthetic construct
  source 1..18: mol_type = other DNA
cccagtttgatggcccca

SEQ ID NO: 7 - DNA, 18 bp, synthetic construct
  source 1..18: mol_type = other DNA
tgcccatgagttccatgc

SEQ ID NO: 8 - DNA, 22 bp, synthetic construct
  source 1..22: mol_type = other DNA
taccacttcacaagtcggaggc

SEQ ID NO: 9 - DNA, 23 bp, synthetic construct
  source 1..23: mol_type = other DNA
ctgcaagtgcatcatcgttgttc

SEQ ID NO: 10 - DNA, 22 bp, synthetic construct
  source 1..22: mol_type = other DNA
ggtgcctatgtctcagcctctt

SEQ ID NO: 11 - DNA, 23 bp, synthetic construct
  source 1..23: mol_type = other DNA
gccatagaactgatgagagggag

SEQ ID NO: 12 - DNA, 22 bp, synthetic construct
  source 1..22: mol_type = other DNA
tggaccttccaggatgaggaca

SEQ ID NO: 13 - DNA, 22 bp, synthetic construct
  source 1..22: mol_type = other DNA
gttcatctcggagcctgtagtg

SEQ ID NO: 14 - DNA, 22 bp, synthetic construct
  source 1..22: mol_type = other DNA
ctcttctgcctgctgcactttg

SEQ ID NO: 15 - DNA, 22 bp, synthetic construct
  source 1..22: mol_type = other DNA
atgggctacaggcttgtcactc

SEQ ID NO: 16 - DNA, 20 bp, synthetic construct
  source 1..20: mol_type = other DNA
gacagccactcacctcttca

SEQ ID NO: 17 - DNA, 20 bp, synthetic construct
  source 1..20: mol_type = other DNA
cctctttgctgctttcacac

SEQ ID NO: 18 - DNA, 22 bp, synthetic construct
  source 1..22: mol_type = other DNA
ccacagaccttccaggagaatg

SEQ ID NO: 19 - DNA, 23 bp, synthetic construct
  source 1..23: mol_type = other DNA
gtgcagttcagtgatcgtacagg

SEQ ID NO: 20 - RNA, 19 nt, synthetic construct
  source 1..19: mol_type = other RNA
gcttagagctcaacccaga

(19) United States
(12) Patent Application Publication
(10) Pub. No.: US 2026/0325607 A1
(43) Pub. Date: Oct. 22, 2026

(54) USE OF VEGF-B PROTEIN OR ACTIVE PEPTIDE OF VEGF-B PROTEIN IN PREPARATION OF DRUG FOR PREVENTING AND/OR TREATING ACUTE ANTERIOR UVEITIS

(71) Applicant: ZHONGSHAN OPHTHALMIC CENTER, SUN YAT-SEN UNIVERSITY, Guangdong (CN)

(72) Inventors: Xuri LI, Guangdong (CN); Weisi LU, Guangdong (CN)

(73) Assignee: ZHONGSHAN OPHTHALMIC CENTER, SUN YAT-SEN UNIVERSITY, Guangdong (CN)

(21) Appl. No.: 19/485,655

(22) PCT Filed: Apr. 29, 2025
(86) PCT No.: PCT/CN2025/092006

§ 371 Date: Nov. 18, 2025

(30) Foreign Application Priority Data
Oct. 9, 2024 (CN) 202411396665.9

(51) Int. Cl. A61K 38/18; A61P 27/02

(52) U.S. Cl. CPC A61K 38/1866; A61P 27/02

(57) ABSTRACT

The present disclosure provides use of a VEGF-B protein and an active peptide of the VEGF-B protein in preparation of a drug for preventing and/or treating acute anterior uveitis (AAU). The VEGF-B protein and the active peptide of the VEGF-B protein provided by the present disclosure have good safety of a patent drug and an anti-AAU effect, can reduce the number of inflammatory cells in eyes and inhibit the expression of inflammatory factors, and can also inhibit the M1 polarization of macrophages. Therefore, the present disclosure provides a new idea for the prevention and treatment of eye diseases, and demonstrates a very promising application prospect in the medical field.

### TECHNICAL FIELD

The present disclosure belongs to the technical field of biomedicine, and specifically relates to use of a vascular endothelial growth factor-B (VEGF-B) protein or an active peptide of the VEGF-B protein in preparation of a drug for preventing and/or treating acute anterior uveitis (AAU).

### RELATED ART

Vascular endothelial growth factor-B (VEGF-B) is a member of the vascular endothelial growth factor (VEGF) family. VEGF-B primarily binds to vascular endothelial growth factor receptor-1 (VEGFR-1) and neuropilin-1 (NRP-1), and is expressed in most tissues and organs. When VEGF-B specifically binds to VEGFR-1, vascular endothelial cells can respond to VEGF-B by increasing the expression levels and activities of plasminogen activator inhibitor-1 (PAI-1) and urokinase-type plasminogen activator (uPA). As a receptor for collapsin/semaphorin, NRP-1 can play a role of guiding neuronal growth cones. NRP-1 can selectively bind to VEGF-B to exert its effect.

Uveitis is an intraocular inflammatory disease primarily affecting the uvea including the iris, ciliary body, and choroid. In severe cases, uveitis may even lead to blindness. Blind patients caused by uveitis account for 10% to 15% of blind patients globally. About 4% to 10% of the blind patients in China are attributed to uveitis. Acute anterior uveitis (AAU) is the most common form of uveitis, with an incidence rate of about 28% to 61%. The clinical manifestations of AAU include ciliary congestion, significant anterior chamber flare, sudden eye pain, eye redness, photophobia, blurred vision, etc. In severe cases, AAU may lead to the formation or exacerbation of posterior synechiae and the complication of glaucoma, resulting in irreversible visual impairment or loss. Drugs clinically used for the treatment of anterior uveitis include: 1) Mydriatics and cycloplegics: Mydriatics and cycloplegics can only alleviate clinical symptoms such as pain and photophobia. 2) Nonsteroidal anti-inflammatory drugs: Nonsteroidal anti-inflammatory drugs are effective merely for specified non-necrotizing anterior scleritis. The systemic administration of nonsteroidal anti-inflammatory drugs will increase the risk of gastrointestinal ulcers. 3) Steroids: Steroids are the major drugs for treating uveitis, and can be administered either topically or systemically. However, steroids have severe side effects, such as ocular side effects including cataracts, elevated intraocular pressure, and glaucoma, and systemic side effects including diabetes, hypertension, peptic ulcers, insomnia, and osteoporosis. 4) Immunomodulation therapies, such as anti-TNF-α, anti-IL-6, and anti-IL-1 therapies: When used in combination with the traditional steroids or cellular immunosuppressants, immunomodulation therapies can achieve prominent efficacy. However, the immunosuppression may cause infection, which is a significant side effect. The current clinical drugs for treating AAU can only control inflammations to some extent, and are associated with severe side effects that may further lead to visual loss. Therefore, there is an urgent need for safe and effective targeted therapies for the prevention and treatment of AAU.

### SUMMARY OF INVENTION

An objective of the present disclosure is to provide use of a VEGF-B protein or an active peptide of the VEGF-B protein in preparation of a drug for preventing and/or treating AAU.

According to a first aspect of the present disclosure, use of a VEGF-B protein or an active peptide of the VEGF-B protein in preparation of a drug for preventing and/or treating AAU is provided. The VEGF-B protein and the active peptide of the VEGF-B protein provided in the present disclosure have a prominent anti-inflammatory effect, and can reduce the number of inflammatory cells in eyes and inhibit the expression of inflammatory factors, thereby playing a role of treating AAU. Thus, the present disclosure provides a new idea for the prevention and treatment of eye diseases, and demonstrates a very promising application prospect in the medical field.

Preferably, the drug for preventing and/or treating AAU is a drug for reducing inflammatory cells in eyes.

Preferably, the inflammatory cells include CD45-positive cells (CD45+ cells).

Preferably, the active peptide of the VEGF-B protein includes at least one selected from the group consisting of peptide 1, peptide 2, peptide 3, peptide 4, and peptide 5. An amino acid sequence of the peptide 1 is shown in SEQ ID NO: 1, an amino acid sequence of the peptide 2 is shown in SEQ ID NO: 2, an amino acid sequence of the peptide 3 is shown in SEQ ID NO: 3, an amino acid sequence of the peptide 4 is shown in SEQ ID NO: 4, and an amino acid sequence of the peptide 5 is shown in SEQ ID NO: 5.

Preferably, the active peptide of the VEGF-B protein includes the peptide 5. Among the active peptides of the VEGF-B protein, the peptide 5 exhibits the strongest anti-inflammatory effect.

According to a second aspect of the present disclosure, use of a VEGF-B protein or an active peptide of the VEGF-B protein in preparation of a drug for inhibiting M1 polarization of a macrophage is provided.

Preferably, the drug for inhibiting M1 polarization of a macrophage is a drug for reducing expression of an inflammatory factor.

Preferably, the inflammatory factor includes at least one selected from the group consisting of TNF-α, IL-6, and IL-1β.

Preferably, the active peptide of the VEGF-B protein includes at least one selected from the group consisting of peptide 1, peptide 2, peptide 3, peptide 4, and peptide 5. An amino acid sequence of the peptide 1 is shown in SEQ ID NO: 1, an amino acid sequence of the peptide 2 is shown in SEQ ID NO: 2, an amino acid sequence of the peptide 3 is shown in SEQ ID NO: 3, an amino acid sequence of the peptide 4 is shown in SEQ ID NO: 4, and an amino acid sequence of the peptide 5 is shown in SEQ ID NO: 5.

Preferably, the active peptide of the VEGF-B protein includes the peptide 5. Among the active peptides of the VEGF-B protein, the peptide 5 exhibits the strongest anti-inflammatory effect.

Preferably, the VEGF-B protein includes VEGF-B167.

According to a third aspect of the present disclosure, a drug including a VEGF-B protein or an active peptide of the VEGF-B protein as an active ingredient is provided.

The present disclosure has the following beneficial effects:

The research in the present disclosure shows that the VEGF-B protein and the active peptide of the VEGF-B protein can reduce the number of inflammatory cells in eyes and inhibit the expression of inflammatory factors, and thus has an anti-AAU effect. The VEGF-B protein and the active peptide of the VEGF-B protein can also inhibit the M1 polarization of macrophages. As a result, the VEGF-B protein and the active peptide of the VEGF-B protein can be used to prepare a drug for preventing and treating AAU or a drug for inhibiting the M1 polarization of macrophages. The present disclosure provides a new idea for the prevention and treatment of eye diseases, and demonstrates a very promising application prospect in the medical field.

### BRIEF DESCRIPTION OF DRAWINGS

FIG. 1 shows mRNA expression levels of VEGF-B in iris-ciliary body complexes of mice in a normal group and an AAU model group.

FIG. 2 shows test results for the promotion of the knockout of a VEGF-B gene on inflammatory responses in AAU, where A shows mRNA expression levels of VEGF-B in iris-ciliary body complexes of VEGF-B gene-knockout mice (Vegf-b^{-/-}) and wild-type (WT) littermates thereof; B shows slit-lamp microscope observations for VEGF-B gene-knockout mice (Vegf-b^{-/-}) and WT littermates thereof after modeling; C shows clinical severity scores for VEGF-B gene-knockout mice (Vegf-b^{-/-}) and WT littermates thereof after modeling; D and E show hematoxylin-eosin staining (H&E staining) and quantification results for eyeball sections from VEGF-B gene-knockout mice (Vegf-b^{-/-}) and WT littermates thereof after modeling; and F and G show fluorescence staining and quantification results for eyeball sections from VEGF-B gene-knockout mice (Vegf-b^{-/-}) and WT littermates thereof after modeling.

FIG. 3 shows test results for the inhibition of a VEGF-B protein on the M1 polarization of macrophages, where A shows relative mRNA expression levels of inflammatory factors TNF-α, IL-6, and IL-1β in THP-1 cells with the expression of VEGF-B knocked down by siRNA (si-VEGF-B) and normal THP-1 cells (si-NC); B shows mRNA expression levels of inflammatory factors TNF-α, IL-6, and IL-1β in peritoneal macrophages from VEGF-B gene-knockout mice (Vegf-b^{-/-}) and WT littermates thereof after a lipopolysaccharide (LPS) treatment; and C, D, and E show mRNA expression levels of inflammatory factors IL-1β, IL-6, and TNF-α in THP-1 cells treated with LPS, bovine serum albumin (BSA), and a VEGF-B protein, respectively.

FIG. 4 shows test results for the specific inhibition of a VEGF-B protein on inflammatory responses.

FIG. 5 shows test results for the inhibition of a VEGF-B protein and active peptides thereof on inflammatory responses, where A shows a specific structural domain of VEGF-B167, and B shows mRNA expression levels of inflammatory factors TNF-α, IL-6, and IL-1β in LPS-stimulated THP-1 cells treated with the VEGF-B protein, peptide 1, peptide 2, peptide 3, peptide 4, and peptide 5, respectively.

FIG. 6 shows test results for the treatment of AAU by a VEGF-B protein, where A shows expression levels of a VEGF-B protein in iris-ciliary body complexes of mice in an AAV-VEGF-B group and an AAV-GFP group after an LPS treatment; B shows slit-lamp microscope observations for mice in an AAV-VEGF-B group and an AAV-GFP group after an LPS treatment; C shows clinical severity scores for mice in an AAV-VEGF-B group and an AAV-GFP group after an LPS treatment; D shows flow cytometry results for mice in an AAV-VEGF-B group and an AAV-GFP group after an LPS treatment; E and F show H&E staining and quantification results for eyeball sections from mice in an AAV-VEGF-B group and an AAV-GFP group after an LPS treatment; and G and H show fluorescence staining and quantification results for eyeball sections from mice in an AAV-VEGF-B group and an AAV-GFP group after an LPS treatment.

### DESCRIPTION OF EMBODIMENTS

The present disclosure is further described below with reference to examples, but is not limited thereto.

In the following examples, the experimental methods used are conventional methods and the reagents and materials used are commercially available, unless otherwise specified.

### Example 1: The Expression of VEGF-B is Correlated with AAU

### 1. Modeling of AAU

Mice (C57 mice purchased from GemPharmatech Co., Ltd., Jiangsu, China) were intravitreally injected with LPS to establish an AAU model group (EIU). Mice without intravitreal injection of LPS were set as a normal group.

A modeling method for EIU was as follows: Mice were intraperitoneally injected with 1% sodium pentobarbital (dose: 10 mL/kg) for anesthesia, with tropicamide for pupillary dilation, procainamide for corneal surface anesthesia, and sodium carboxymethyl cellulose for corneal dryness prevention. Mice were intravitreally injected with LPS (1 L/eye, 12.5 ng) through a puncture hole with a diameter of 0.2 mm behind a corneal limbus using a sterilized 5 μL syringe (Hamilton, 7633-01) and 33-gauge blunt needle (Hamilton, 7803-05).

After eyes of the mice were injected with LPS for modeling, the mice underwent symptoms including lacrimation, ocular discharge, miosis, ciliary flush, and edema, indicating the successful establishment of AAU.
2. Detection of mRNA expression levels of VEGF-B in iris-ciliary body complexes of mice in the normal group and the model group by quantitative reverse transcription polymerase chain reaction (qRT-PCR)
1) An iris-ciliary body complex was collected from mice after the modeling, washed once with phosphate buffered saline (PBS), lysed with 500 μL of Trizol, and transferred to a 1.5 mL centrifuge tube;
2) 100 μL of chloroform was added, and a resulting mixture was vigorously shaken for 20 s, allowed to stand for 3 min, and then centrifuged for 10 min at 12,000 rpm and 4 ° C to produce a first supernatant;
3) The first supernatant was transferred to a new centrifuge tube (without disturbing an intermediate layer). Isopropyl alcohol was added to the first supernatant in a volume ratio of 1:1, and a resulting mixed solution was thoroughly mixed, allowed to stand for 10 min, and then centrifuged at 4 ° C for 10 min to produce a second supernatant;
4) The second supernatant was discarded, and 1 mL of 75% ethanol was added;
5) Centrifugation was conducted at 4 ° C for 5 min to produce a third supernatant. The third supernatant was discarded, and 1 mL of 75% ethanol was added. Centrifugation was conducted at 4 ° C for 5 min to produce a fourth supernatant. The fourth supernatant was discarded, and the centrifuge tube was inverted on clean absorbent paper. After a white precipitate at a bottom of the centrifuge tube became transparent, 50 μL of ddH₂O was added, and a resulting system was stored at −20 ° C;
6) Total RNA was obtained, and reverse-transcribed using a FastKing RT kit with gDNase (TIANGEN) kit to produce cDNA;
7) With the synthesized cDNA as a template, an mRNA expression level of VEGF-B was detected on an ABI QuantStudio 6 Flex device (Life Technologies) with reference to instructions of an SYBR Green (ROCBE) kit;
8) With 185 rRNA as an internal reference gene, an expression level of each gene was calculated by the delta-delta Ct method. Primer sequences for each gene were acquired from the Primerbank website.

TABLE 1
qPCR primer sequences for each gene

| Gene | Primer sequence (5′-3′) | Sequence No. |
|---|---|---|
| Ms-VEGF-B-F | CCCAGTTTGATGGCCCCA | SEQ ID NO: 6 |
| Ms-VEGF-B-R | TGCCCATGAGTTCCATGC | SEQ ID NO: 7 |
| Ms-IL-6-F | TACCACTTCACAAGTCGGAGGC | SEQ ID NO: 8 |
| Ms-IL-6-R | CTGCAAGTGCATCATCGTTGTTC | SEQ ID NO: 9 |
| Ms-Tnf-a-F | GGTGCCTATGTCTCAGCCTCTT | SEQ ID NO: 10 |
| Ms-Tnf-a-R | GCCATAGAACTGATGAGAGGGAG | SEQ ID NO: 11 |
| Ms-IL-1b-F | TGGACCTTCCAGGATGAGGACA | SEQ ID NO: 12 |
| Ms-IL-1b-R | GTTCATCTCGGAGCCTGTAGTG | SEQ ID NO: 13 |
| hu-TNF-a-F | CTCTTCTGCCTGCTGCACTTTG | SEQ ID NO: 14 |
| hu-TNF-a-R | ATGGGCTACAGGCTTGTCACTC | SEQ ID NO: 15 |
| hu-IL-6-F | GACAGCCACTCACCTCTTCA | SEQ ID NO: 16 |
| hu-IL-6-R | CCTCTTTGCTGCTTTCACAC | SEQ ID NO: 17 |
| hu-IL-1β-F | CCACAGACCTTCCAGGAGAATG | SEQ ID NO: 18 |
| hu-IL-1β-R | GTGCAGTTCAGTGATCGTACAGG | SEQ ID NO: 19 |

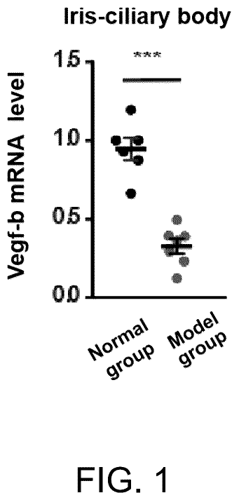

qRT-PCR test results are shown in FIG. 1. As shown in FIG. 1, mice in a model group have a significantly-lower mRNA expression level of VEGF-B in an iris-ciliary body complex than mice in the normal group, indicating that the expression of VEGF-B is correlated with AAU.

### Example 2: The Knockout of a VEGF-B Gene can Exacerbate Inflammatory Responses in AAU

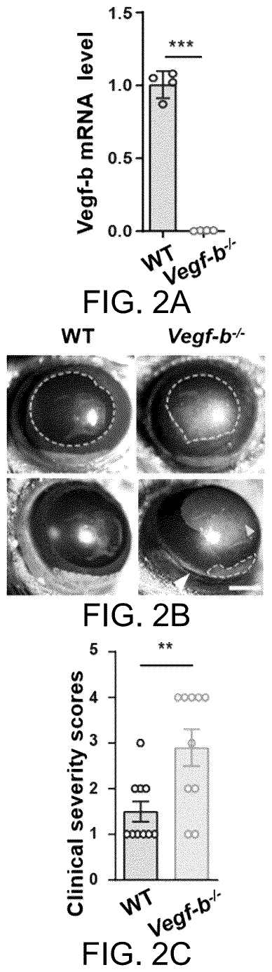

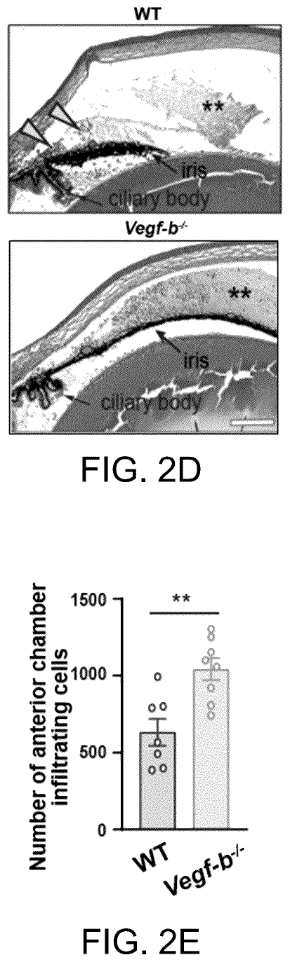

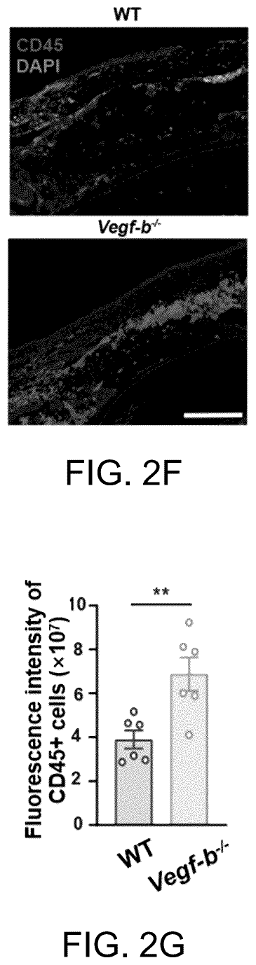

1. Modeling of AAU: VEGF-B gene-knockout mice (Vegf-b^{-/-}) and WT littermates thereof were intravitreally injected with LPS (1 μL/eye, 12.5 ng). The VEGF-B gene-knockout mice (Vegf-b^{-/-}) and the WT littermates thereof were constructed by the Jackson Laboratory through the colonial breeding of parents with genotypes of het×het.
2. mRNA expression levels of VEGF-B in iris-ciliary body complexes of the VEGF-B gene-knockout mice (Vegf-b^{-/-}) and the WT littermates thereof were detected by qRT-PCR. qRT-PCR test results are shown in A of FIG. 2. As shown in A of FIG. 2, the VEGF-B gene-knockout mice (Vegf-b^{-/-}) have a significantly-lower relative mRNA expression level of VEGF-B in an iris-ciliary body complex than the WT littermates.
3. Inflammatory manifestations in anterior segments of the VEGF-B gene-knockout mice (Vegf-b^{-/-}) and the WT littermates thereof after modeling were observed under a slit-lamp microscope.

Slit-lamp microscope observations of anterior segments of the VEGF-B gene-knockout mice (Vegf-b^{-/-}) and the WT littermates thereof after modeling are shown in B of FIG. 2 (scale bar: 200 μm). As shown in B of FIG. 2, compared with the WT littermates, the VEGF-B gene-knockout mice (Vegf-b^{-/-}) exhibit exacerbated inflammatory manifestations, including aggravated miosis due to posterior synechiae (circled by a white dashed line); adhesion of increased inflammatory cells and pigment cells to the posterior corneal surface (indicated by a yellow arrow); intensified ciliary congestion (indicated by a white arrow); and aggravated hypopyon in the anterior chamber (circled by a yellow dashed line).
4. The VEGF-B gene-knockout mice (Vegf-b^{-/-}) and the WT littermates thereof after modeling were scored according to clinical scoring criteria for AAU:

Clinical severity scores for the VEGF-B gene-knockout mice (Vegf-b^{-/-}) and the WT littermates thereof after modeling are shown in C of FIG. 2. As shown in C of FIG. 2, the VEGF-B gene-knockout mice (Vegf-b^{-/-}) have higher clinical severity scores and more severe inflammatory manifestations than the WT littermates.
5. Eyeball sections from the VEGF-B gene-knockout mice (Vegf-b^{-/-}) and the WT littermates thereof after modeling were subjected to H&E staining and immunofluorescence staining.

Operations of the H&E staining were as follows: Eyeball sections were deparaffinized with first xylene (SCRC, 10023418) for 20 min, then with second xylene (SCRC, 10023418) for 20 min, then with first 100% ethanol (SCRC, 100092683) for 5 min, then with second 100% ethanol (SCRC, 100092683) for 5 min, and finally with 75% ethanol for 5 min. Deparaffinized eyeball sections were stained with a hematoxylin solution (Servicebio, G1003) for 5 min, rinsed with tap water, then differentiated with a hematoxylin differentiation solution (Servicebio, G1003), rinsed with tap water, then treated with hematoxylin-scott Tap blue (Servicebio, G1003), rinsed with tap water, treated with 85% ethanol for 5 min and then with 95% ethanol for 5 min, stained with an eosin dye (Servicebio, G1003) for 5 min, dehydrated, mounted with a neutral gum (SCRC, 10004160), and photographed with an AX10 imager Z2 (Zeiss) microscope.

Operations of the immunofluorescence staining were as follows: Eyeball sections were incubated in 0.5% Triton X-100 (Sigma, X100) prepared with 1×PBS for 15 min, then blocked with 5% normal goat serum for 1 h, then incubated with a primary antibody at 4 ° C overnight, washed three times with 1×PBS, incubated with a secondary antibody at room temperature for 1 h, subjected to nuclear staining with 4′,6-diamidino-2-phenylindole (DAPI) for 5 min (Sigma, D9542), and imaged with an AX10 imager Z2 (Zeiss) microscope. Analysis was conducted with ZEN 2012 (Zeiss) and ImageJ.

H&E staining results for the eyeball sections from the VEGF-B gene-knockout mice (Vegf-b^{-/-}) and the WT littermates thereof are shown in D (scale bar: 50 μm) and E of FIG. 2. As shown in D and E of FIG. 2, compared to the WT littermates, the VEGF-B gene-knockout mice (Vegf-b^{-/-}) have increased CD45-positive cell infiltration (indicated by a yellow arrow) and increased fibrin exudation (indicated by **) in the anterior chamber.

Immunofluorescence staining results for the eyeball sections from the VEGF-B gene-knockout mice (Vegf-b^{-/-}) and the WT littermates thereof are shown in F (scale bar: 50 μm) and G of FIG. 2. As shown in F and G of FIG. 2, the VEGF-B gene-knockout mice (Vegf-b^{-/-}) have a significantly-larger number of CD45-positive cells than the WT littermates.

These results demonstrate that the loss of the VEGF-B function exacerbates the inflammatory responses in AAU.

### Example 3: a VEGF-B Protein has an Inhibitory Effect on the M1 Polarization of Macrophages

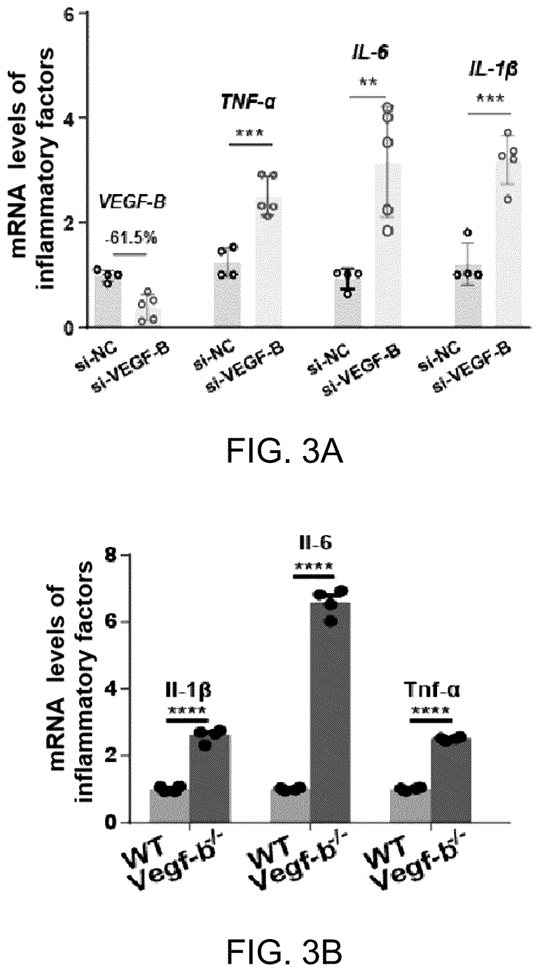

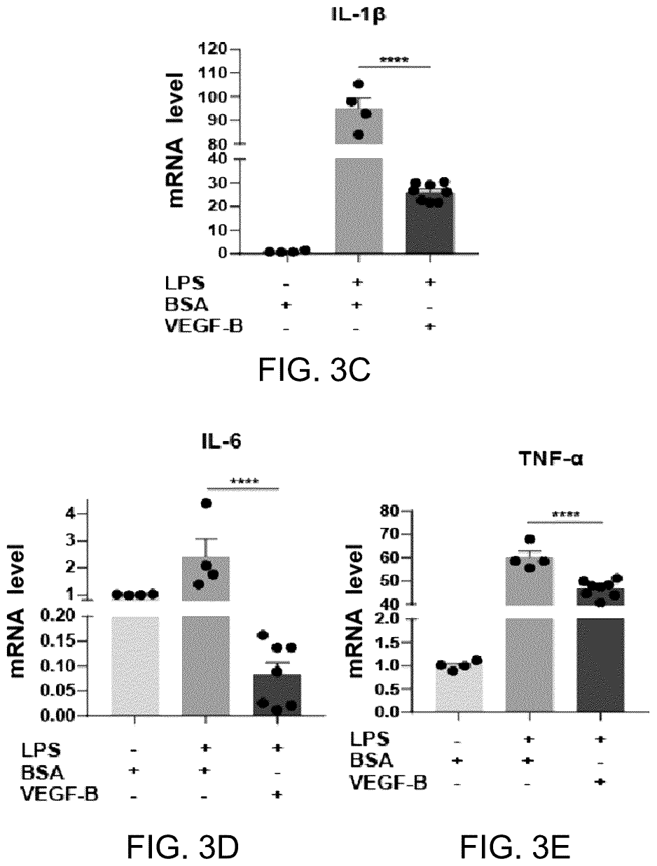

1. The expression of VEGF-B in THP-1 cells (purchased from Zhongqiao Xinzhou Biotechnology Co., Ltd., Item No.: ZQ0086) was knocked down by siRNA. Expression levels of inflammatory factors TNF-α, IL-6, and IL-1β in THP-1 cells with the expression of VEGF-B knocked down by siRNA (si-VEGF-B) and in normal THP-1 cells (si-NC) were determined by qRT-PCR. qRT-PCR test results are shown in A of FIG. 3. As shown in A of FIG. 3, the THP-1 cells with the expression of VEGF-B knocked down by siRNA (si-VEGF-B) have significantly-higher expression levels of inflammatory factors TNF-α, IL-6, and IL-1β than the normal THP-1 cells (si-NC). These results indicate that the knockdown of the expression of the VEGF-B protein can significantly promote the expression of the inflammatory factors in cells, thereby driving the polarization of macrophages toward the M1 subtype.

A method for knocking down the expression of VEGF-B in THP-1 cells by siRNA was as follows:
(1) 1×10⁶ THP-1 cells were inoculated into each well of a 6-well plate. After induced adhesion, a transfection system was prepared according to the following protocol:
Solution A: 6 μL of RNAiMax was added to 100 μL of Opti-MEM to produce a first mixture, and the first mixture was fully mixed gently and then allowed to stand at room temperature for 5 min.
Solution B: 3 μL of siRNA (a sequence of the siRNA: GCTTAGAGCTCAACCCAGA) at a concentration of 20 M was added to 100 μL of Opti-MEM to produce a second mixture, and the second mixture was fully mixed gently and then allowed to stand at room temperature for 5 min.
(2) The solution A was gently mixed with the solution B thoroughly to produce a mixed solution, and the mixed solution was allowed to stand at room temperature for 10 min to produce a transfection solution.
(3) The transfection solution obtained in the step (2) was diluted with Opti-MEM to 1.5 mL, and then added to the 6-well plate.
4) The 6-well plate was incubated in an incubator for 6 h, and then the original medium was replaced with a complete medium. 48 h later, the subsequent experiments were conducted.
2. Peritoneal macrophages from the VEGF-B gene-knockout mice (Vegf-b^{-/-}) and the WT littermates thereof were treated with LPS. mRNA expression levels of inflammatory factors TNF-α, IL-6, and IL-1β in LPS-stimulated peritoneal macrophages from the VEGF-B gene-knockout mice (Vegf-b^{-/-}) and the WT littermates thereof were determined by qRT-PCR. qRT-PCR test results are shown in B of FIG. 3. As shown in B of FIG. 3, LPS-stimulated peritoneal macrophages from the VEGF-B gene-knockout mice (Vegf-b^{-/-}) demonstrate significantly-higher mRNA expression levels of inflammatory factors TNF-α, IL-6, and IL-1β than LPS-stimulated peritoneal macrophages from the WT littermates.

A method for collection and culture of peritoneal macrophages (PCMs) from mice was as follows:
(1) Preparation of a fluid thioglycollate (FTG) solution:
4 g of FTG (FTG medium: manufacturer: Sigma; Item No.: T9032; and specification: 1 kg) and 100 mL of H₂O were mixed, autoclaved, and then refrigerated at 4 ° C for later use.
(2) 6-8 week-old mice were intraperitoneally injected with the FTG solution at 2 mL/mouse.

A mouse was restrained by the left hand with the abdomen facing upward. A syringe needle was inserted by the right hand subcutaneously into the lower left abdomen. The syringe needle was pushed forward by 0.5 cm, then allowed to penetrate through the abdominal muscle at an angle of 45°, and secured. The FTG solution was injected slowly. To avoid the damage to internal organs, the mouse was maintained in a head-down tilt to make the internal organs move toward the upper abdomen.
(3) 2 d later, the mouse was sacrificed, and peritoneal macrophages were collected.
1) The mouse was sacrificed through cervical dislocation;
2) The abdomen was soaked in or scrubbed with 75% alcohol, and then placed on a workbench with a ventral side facing upward;
3) The skin of the lower abdomen of the mouse was lifted with forceps, and a small incision was created. The skin was torn open upward and downward to fully expose the peritoneum;
4) 8 mL of a 1640 medium was injected by a 10 mL syringe, and the abdomen of the mouse was appropriately shaken and massaged;
5) About 6 mL of a peritoneal fluid was drawn out by a small needle with a tip facing upward and avoiding intestines and fats;
6) The peritoneal lavage was repeated once (the steps 4 and 5);
7) A resulting cell suspension was centrifuged, and a resulting supernatant was discarded;
8) Cells were resuspended with an appropriate amount of a 1640 complete medium and counted;
9) After the counting, the cells were inoculated into a 6-well plate at 2×10⁶ cells/well;
10) After being cultured at 37 ° C for 2 h, the cells were washed once with a 1640 medium. Then the medium was replaced to remove non-adherent cells.
3. THP-1 cells were treated with LPS, and a VEGF-B recombinant protein was added to the cells. mRNA expression levels of inflammatory factors TNF-α, IL-6, and IL-1β in THP-1 cells treated with LPS were determined by qRT-PCR. qRT-PCR test results are shown in C to E of FIG. 3. As shown in C to E of FIG. 3, the addition of the control BSA to the LPS-treated THP-1 cells leads to relatively-high expression levels of inflammatory factors TNF-α, IL-6, and IL-1β. In contrast, the addition of the VEGF-B recombinant protein to the LPS-treated THP-1 cells leads to relatively-low expression levels of inflammatory factors TNF-α, IL-6, and IL-1β. It can be seen that the VEGF-B protein can effectively inhibit the polarization of macrophages toward the pro-inflammatory M1 subtype.

### Example 4: a VEGF-B Protein Exhibits Specific Inhibition on Inflammatory Responses

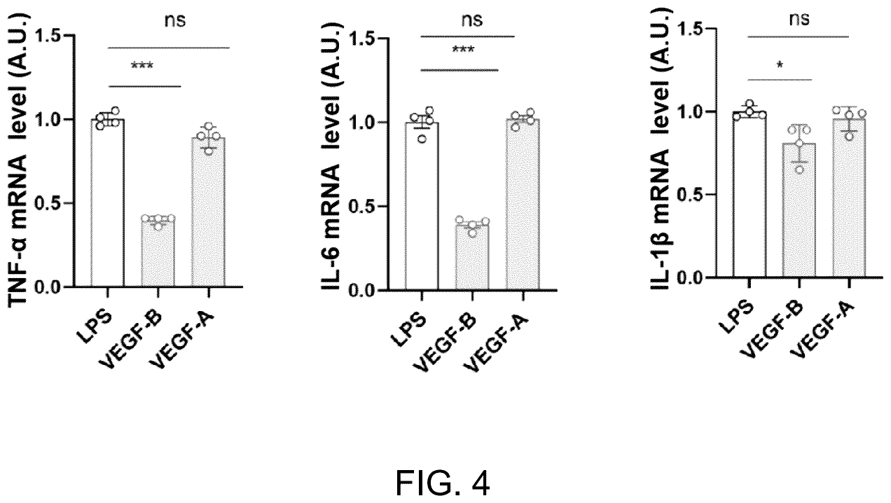

1. Bone marrow-derived macrophages (BMDMs) were collected from WT mice: A mouse was anaesthetized, then sacrificed through cervical dislocation, and then placed in a beaker with sufficient 75% ethanol for 5 min to allow disinfection. The tibia and femur were isolated from the mouse on a sterile operating bench. The bone marrow was flushed out from the tibia and femur with a pre-cooled medium. The tibia and femur were repeatedly rinsed three times until no obvious redness could be observed inside the bones. A bone marrow cell-containing medium was repeatedly pipetted up and down with a 5 mL pipette to make cell aggregates dispersed. Cells were then filtered through a 70 m cell strainer, transferred to a 15 mL centrifuge tube, and centrifuged at 1,500 rpm/min for 5 min to produce a first supernatant. The first supernatant was discarded, and cells were resuspended in a red blood cell lysis buffer, allowed to stand for 5 min, and centrifuged at 1,500 rpm/min for 5 min to produce a second supernatant. The second supernatant was discarded, and cells were resuspended in a cold prepared BMDM induction medium and then inoculated (2×10⁶ viable cells/dish). On day 3 of culture, a half of the BMDM induction medium was replaced. On day 5 of culture, the medium was completely replaced. On day 7 of culture, resulting cells were ready for subsequent experiments.
2. Mouse BMDMs were stimulated with LPS for 6 h. A VEGF-B recombinant protein (100-20B, Peprotech) and a VEGF-A recombinant protein (100-20, Peprotech) were separately added to the cells at 1.5 L/well with a final protein concentration of 100 ng/L, and the cells were then incubated for 6 h. mRNA expression levels of inflammatory factors TNF-α, IL-6, and IL-1β in mouse BMDMs were detected by qRT-PCR. qRT-PCR test results are shown in FIG. 4. As shown in FIG. 4, the VEGF-B protein demonstrates a significant inhibitory effect on the inflammatory responses, but the VEGF-A protein demonstrates no obvious inhibitory effect on the inflammatory responses. It can be seen that the inhibition of the VEGF-B protein on the inflammatory responses is specific.

### Example 5: Specific Structural Domains of the VEGF-B Protein to Exert an Anti-Inflammatory Effect

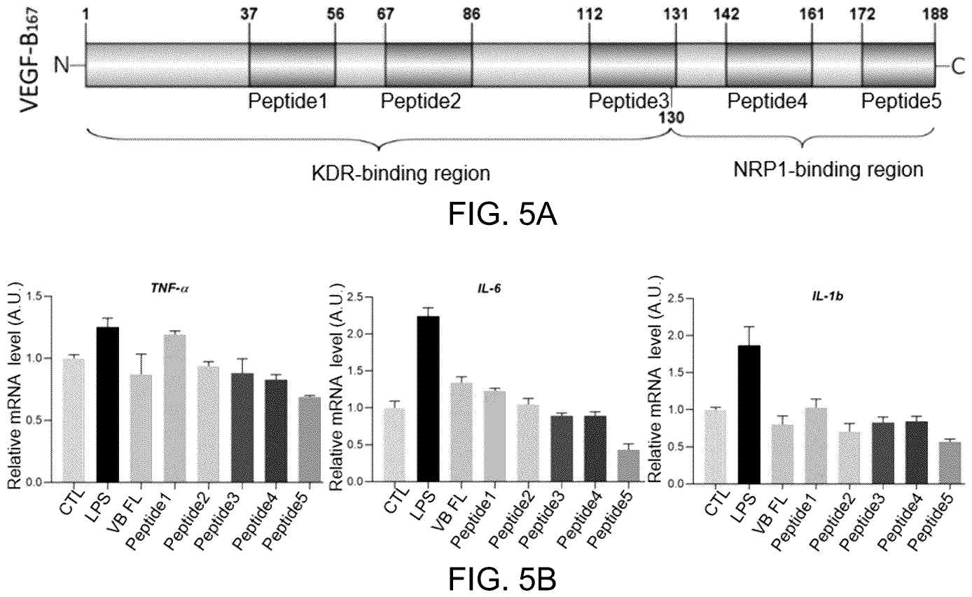

1. The specific structural domains of the active peptide (Peptide1, Peptide2, Peptide3, Peptide4, and Peptide5) derived from the VEGF-B167 protein are shown in A of FIG. 5. Sequences of the active peptides are listed in Table 2. As shown in A of FIG. 5, the peptide 1, the peptide 2, and the peptide 3 are located in a VEGFR1-binding region (KDR-binding region), while the peptide 4 and the peptide 5 are located in an NRP1-binding region.

TABLE 2
Sequences of the active peptides of the VEGF-B167
protein

| Peptide | Sequence | Sequence No. |
|---|---|---|
| Peptide 1 | PVSQPDAPGHQRKVVSWIDV | SEQ ID NO: 1 |
| Peptide 2 | SWIDVYTRATCQPREVVVPL | SEQ ID NO: 2 |
| Peptide 3 | VVVPLTVELMGTVAKQLVPS | SEQ ID NO: 3 |
| Peptide 4 | QLVPSCVTVQRCGGCCPDDG | SEQ ID NO: 4 |
| Peptide 5 | CPDDGLECVPTGQHQVRMQI | SEQ ID NO: 5 |

2. Normal THP-1 cells were set as a control group (CTL group). THP-1 cells stimulated with LPS were set as an LPS group. A full-length VEGF-B protein was added to cells stimulated with LPS for 6 h to establish a VB FL group. The peptide 1 as an active peptide of the VEGF-B protein was added to cells stimulated with LPS for 6 h to establish a peptide 1 group. The peptide 2 was added to cells stimulated with LPS for 6 h to establish a peptide 2 group. The peptide 3 was added to cells stimulated with LPS for 6 h to establish a peptide 3 group. The peptide 4 was added to cells stimulated with LPS for 6 h to establish a peptide 4 group. The peptide 5 was added to cells stimulated with LPS for 6 h to establish a peptide 5 group.

mRNA expression levels of inflammatory factors TNF-α, IL-6, and IL-1β in the CTL group, the LPS group, the VB FL group, the peptide 1 group, the peptide 2 group, the peptide 3 group, the peptide 4 group, and the peptide 5 group were detected by qRT-PCR. qRT-PCR test results are shown in B of FIG. 5. As shown in B of FIG. 5, compared to the CTL group, expression levels of the inflammatory factors TNF-α, IL-6, and IL-1β are significantly enhanced in the LPS group. Compared to the LPS group, expression levels of the inflammatory factors TNF-α, IL-6, and IL-1β decrease in cells treated with the full-length VEGF-B protein, the peptide 1, the peptide 2, the peptide 3, the peptide 4, and the peptide 5 (a dose of each protein is 1.5 L/well, and a final protein concentration is 100 ng/L). It can be seen that the full-length VEGF-B protein, the peptide 1, the peptide 2, the peptide 3, the peptide 4, and the peptide 5 all exhibit specified anti-inflammatory effects, and the peptide 5 located in the NRP1-binding region demonstrates the most significant anti-inflammatory effect.

### Example 6: A VEGF-B Protein Exhibits a Therapeutic Effect for AAU

1. Mice were intravitreally injected with an adeno-associated virus expressing a VEGF-B167 protein (purchased from Shandong WZ Biosciences Inc.) to establish an AAV-VEGF-B group. Mice were intravitreally injected with an adeno-associated virus expressing GFP to establish an AAV-GFP group.
2. Expression levels of the VEGF-B protein in iris-ciliary body complexes of mice from the AAV-VEGF-B group and the AAV-GFP group were detected by Western blotting.

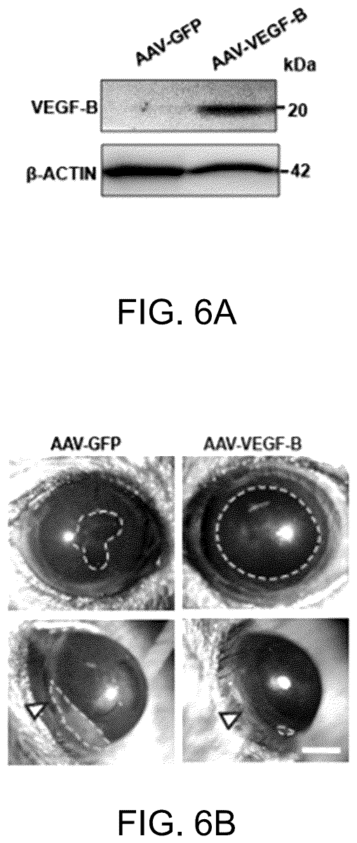

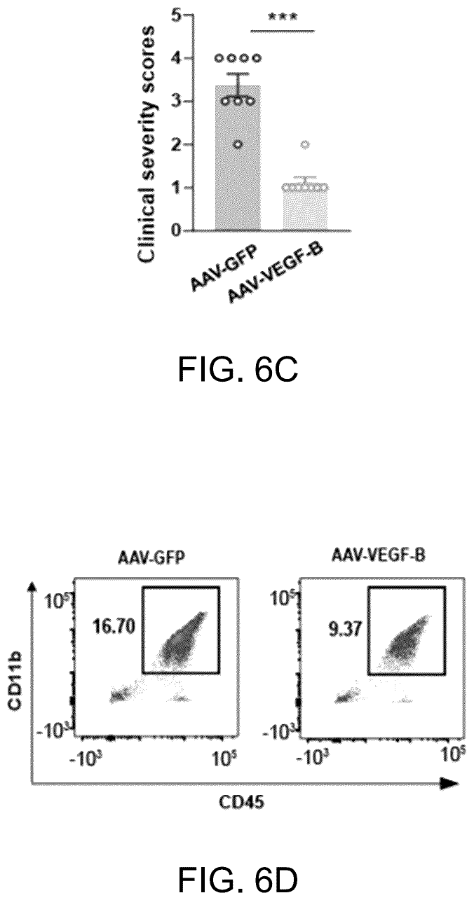

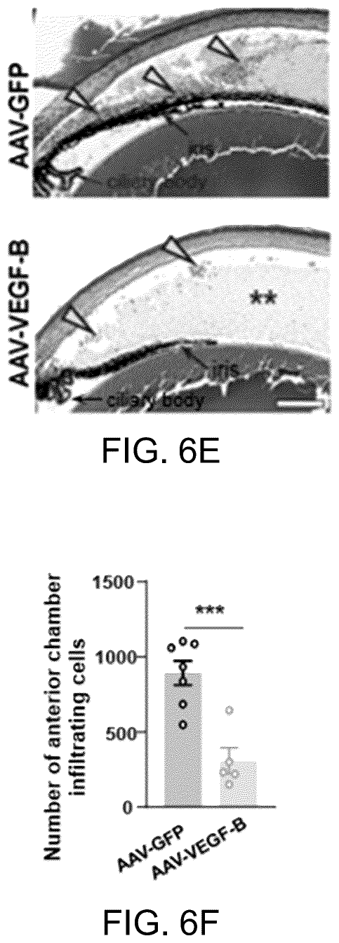

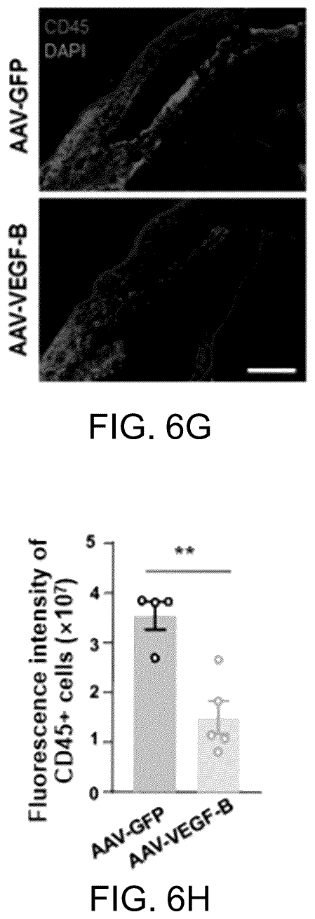

Western blotting test results are shown in A of FIG. 6. As shown in A of FIG. 6, compared to the AAV-GFP group, the VEGF-B protein is over-expressed in iris-ciliary body complexes of mice from the AAV-VEGF-B group.
3. Modeling of AAU: Mice in both the AAV-VEGF-B group and the AAV-GFP group were treated with LPS.
4. Inflammatory manifestations in anterior segments of mice from the AAV-VEGF-B group and the AAV-GFP group after modeling were observed under a slit-lamp microscope.

Slit-lamp microscope observations of anterior segments of mice from the AAV-VEGF-B group and the AAV-GFP group after modeling are shown in B of FIG. 6 (scale bar: 200 μm). As shown in B of FIG. 6, mice in the AAV-GFP group after modeling exhibit severe inflammatory manifestations, including aggravated miosis due to posterior synechiae (circled by a white dashed line), intensified ciliary congestion (indicated by a white arrow), and aggravated hypopyon in the anterior chamber (circled by a yellow dashed line). In contrast, inflammatory manifestations in mice of the AAV-VEGF-B group after modeling are significantly improved, including a normal pupil (indicated by a white dashed line), a clear and transparent cornea without obvious ciliary congestion (indicated by a white arrow), and no significant hypopyon in the anterior chamber (indicated by a yellow dashed line).
5. Mice in the AAV-VEGF-B group and the AAV-GFP group after modeling were scored according to clinical scoring criteria for AAU.

Clinical severity scores are shown in C of FIG. 6. As shown in C of FIG. 6, mice in the AAV-VEGF-B group have lower clinical severity scores and milder inflammatory manifestations than mice in the AAV-GFP group.
6. Eyes of mice in the AAV-VEGF-B group and the AAV-GFP group after modeling were subjected to flow cytometry assay.
(1) Preparation of a single-cell suspension from a mouse anterior segment tissue: Mouse eyeballs were immersed in RPIM-1640 including 10% of FBS. Under a microscope, periocular muscles and fasciae were removed by ophthalmic scissors. A circular incision was created along a corneoscleral limbus, and anterior and posterior chamber tissues were isolated. Infiltrating cells in a vitreous chamber were gently drawn out by a 200 L pipette tip, and a crystalline lens, a retina, and a choroid were taken out. An iris was separated from a ciliary body-cornea complex by forceps, gently torn into small pieces, and the resulting mouse anterior chamber tissues-containing medium was transferred to a 15 mL centrifuge tube. Centrifugation was conducted at 50 g for 1 min to produce a medium supernatant and a remaining tissue. The medium supernatant was transferred to a second 15 mL centrifuge tube. The remaining tissue was washed twice with PBS. Then, 500 L of 7 mg/mL collagenase I was added, and digestion was allowed at 37 ° C for 20 min. 3 mg/mL DNase I was added, and digestion was continued at 37 ° C for 10 min. The medium supernatant collected previously was added for enzyme inactivation. A resulting tissue solution was gently pipetted up and down with a 1 mL pipette tip, and then filtered through 70 m and 40 m cell strainers successively to obtain a single-cell suspension.
(2) Flow cytometry assay: The single-cell suspension prepared above was washed twice with PBS including 1% of BSA, then incubated with a CD16/32 antibody (1:200) on ice for 20 min, and then incubated with a target antibody on ice for 30 min in the dark. Cells were washed three times with PBS including 1% of BSA, then stained with propidium iodide (PI), then washed three times with PBS including 1% of BSA, resuspended, and then loaded on a flow cytometer for testing. Data was analyzed using flowJo software.

Flow cytometry results of single-cell suspensions from mouse anterior chamber tissues (including a cornea, an iris, a ciliary body, and infiltrating cells) are shown in D of FIG. 6. As shown in D of FIG. 6, mice in the AAV-VEGF-B group have a significantly-lower proportion of inflammatory cells in the anterior chamber than mice in the AAV-GFP group.
7. Eyeball sections from mice in the AAV-VEGF-B group and the AAV-GFP group after modeling were subjected to H&E staining and immunofluorescence staining.

H&E staining results of the eyeball sections from the mice in the AAV-VEGF-B group and the AAV-GFP group are shown in E (scale bar: 50 μm) and F of FIG. 6. As shown in E and F of FIG. 6, mice in the AAV-VEGF-B group have the CD45-positive cell infiltration and fibrin exudation (indicated by **) significantly reduced in the anterior chamber.

Immunofluorescence staining results of the eyeball sections from the mice in the AAV-VEGF-B group and the AAV-GFP group are shown in G (scale bar: 50 μm) and H of FIG. 6. As shown in G and H of FIG. 6, mice in the AAV-VEGF-B group have a significantly-smaller number of infiltrating CD45-positive cells in the anterior chamber than mice in the AAV-GFP group. Consequently, it can be known that the VEGF-B protein can effectively inhibit the infiltration of inflammatory cells in eyes and protect the integrity of the blood-aqueous barrier, indicating a therapeutic effect of the VEGF-B protein for AAU.

The above examples are merely intended to illustrate the technical solutions of the present disclosure rather than to limit the protection scope of the present disclosure. Although the present disclosure has been described in detail with reference to the above examples, those of ordinary skill in the art should understand that modifications or equivalent replacements may be made to the technical solutions of the present disclosure, but such modifications or replacements shall fall within the protection scope of the present disclosure.

1. Use of a VEGF-B protein or an active peptide of the VEGF-B protein in preparation of a drug for preventing and/or treating acute anterior uveitis (AAU), wherein the active peptide of the VEGF-B protein comprises at least one selected from a group consisting of peptide 1, peptide 2, peptide 3, peptide 4, and peptide 5; and
an amino acid sequence of the peptide 1 is shown in SEQ ID NO: 1,
an amino acid sequence of the peptide 2 is shown in SEQ ID NO: 2,
an amino acid sequence of the peptide 3 is shown in SEQ ID NO: 3,
an amino acid sequence of the peptide 4 is shown in SEQ ID NO: 4, and an amino acid sequence of the peptide 5 is shown in SEQ ID NO: 5.

2. The use according to claim 1, wherein the drug for preventing and/or treating AAU is a drug for reducing inflammatory cells in eyes.

3. The use according to claim 2, wherein the inflammatory cells in eyes comprise CD45-positive cells (CD45+ cells).

4. The use according to claim 1, wherein the active peptide of the VEGF-B protein comprises the peptide 5.

5. Use of a VEGF-B protein or an active peptide of the VEGF-B protein in preparation of a drug for inhibiting M1 polarization of a macrophage wherein the active peptide of the VEGF-B protein comprises at least one selected from a group consisting of peptide 1, peptide 2, peptide 3, peptide 4, and peptide 5; and
an amino acid sequence of the peptide 1 is shown in SEQ ID NO: 1,
an amino acid sequence of the peptide 2 is shown in SEQ ID NO: 2,
an amino acid sequence of the peptide 3 is shown in SEQ ID NO: 3,
an amino acid sequence of the peptide 4 is shown in SEQ ID NO: 4, and
an amino acid sequence of the peptide 5 is shown in SEQ ID NO: 5.

6. The use according to claim 5, wherein the drug for inhibiting M1 polarization of a macrophage is a drug for reducing expression of an inflammatory factor.

7. The use according to claim 6, wherein the inflammatory factor comprises at least one selected from a group consisting of TNF-α, IL-6, and IL-1β.

8. A drug, comprising a VEGF-B protein or an active peptide of the VEGF-B protein as an active ingredient, wherein the active peptide of the VEGF-B protein comprises at least one selected from a group consisting of peptide 1, peptide 2, peptide 3, peptide 4, and peptide 5; and
an amino acid sequence of the peptide 1 is shown in SEQ ID NO: 1,
an amino acid sequence of the peptide 2 is shown in SEQ ID NO: 2,
an amino acid sequence of the peptide 3 is shown in SEQ ID NO: 3,
an amino acid sequence of the peptide 4 is shown in SEQ ID NO: 4, and
an amino acid sequence of the peptide 5 is shown in SEQ ID NO: 5.
